(19) United States
(12) Patent Application Publication
(10) Pub. No.: US 2009/0299479 A1
(43) Pub. Date: Dec. 3, 2009

(54) SUTURE GUIDED IMPLANT

(76) Inventors: Robert J. Jones, Austin, TX (US); Mukund Gundanna, Bryan, TX (US)

Correspondence Address: JOHNSON & ASSOCIATES, PO BOX 90698, AUSTIN, TX, 78709-0698, US

(21) Appl. No.: 12/254,715

(22) Filed: Oct. 20, 2008

Related U.S. Application Data
(60) Provisional application No. 60/981,376, filed on Oct. 19, 2007.

(51) Int. Cl. A61F 2/44; A61L 17/00

(52) U.S. Cl. 623/17.16; 606/228

(57) ABSTRACT

Methods and apparatuses are disclosed relating to surgical implants having one or more strands extending from the implant to assist with the installation of the implants into patients. In one example, the strand may be used during installation to provide a constant reference to the trailing edge of the implant in situ. In another example, the strand may be used during installation to provide guidance to the implant for instrumentation. In another example, the strand may be used during installation to provide the ability to pull back on the implant. In another example, the strand allows for novel designs of installation and removal instruments.

### CROSS REFERENCE TO RELATED APPLICATIONS

This application claims priority under 35 U.S.C. § 119 to co-pending, commonly owned U.S. provisional patent application Ser. No. 60/981,376 filed on Oct. 19, 2007, entitled “SUTURE GUIDED TLIF AND ASSOCIATED METHODS”, which is incorporated by reference herein.

### FIELD OF THE INVENTION

This disclosure relates to the field of surgical implants. In particular, this disclosure is drawn to a suture guided surgical implants.

### BACKGROUND OF THE INVENTION

During the installation of surgical implants, it is usually desired to minimize the invasiveness of the surgery. Some surgical procedures avoid open invasive surgery in favor of minimally invasive surgical procedures. Examples of minimally invasive surgical procedures include the use of laparoscopic devices and remote-control manipulation of instruments with indirect observation of the procedure through an endoscope or similar device. Such procedures are typically carried out through the skin.

When a surgical implant is installed during a minimally invasive surgery, a surgeon may not be able to directly see the surgical implant. It may be a challenge to determine the exact location of the implant during the procedure. I may also be difficult to guide instrumentation devices to the implant after the implant is inside the body.

### SUMMARY OF THE INVENTION

One embodiment of an apparatus provides a surgical implant including an implant, and a strand coupled to and extending from the implant, wherein the strand is configured to provide guidance for surgical instruments that are used during the installation of the implant.

One embodiment includes a spinal fusion device including a spacer configured to be placed between adjacent vertebrae, and a strand coupled to the implant, wherein the strand is configured to provide guidance for surgical instruments that are used during the installation of the implant.

Another embodiment provides a method of installing a surgical implant including providing an implant coupled to a strand that extends from the implant, using the strand to guide one or more instrumentation devices to the implant, and using the one or more instrumentation devices to position the implant where desired.

Other features and advantages of the present invention will be apparent from the accompanying drawings and from the detailed description that follows below.

### BRIEF DESCRIPTION OF THE DRAWINGS

The present invention is illustrated by way of example and not limitation in the figures of the accompanying drawings, in which like references indicate similar elements and in which:

FIG. 1 is an isometric view of a surgical device, including an implant and a strand extending from the implant.

FIG. 2 is an enlarged isometric view of the implant shown in FIG. 1.

FIG. 3 is an enlarged front view of the implant shown in FIG. 1.

FIG. 4 is an enlarged side view of the implant shown in FIG. 1.

FIG. 5 is an enlarged top view of the implant shown in FIG. 1.

FIG. 6 is a partial enlarged view of the strand shown in FIG. 1.

FIG. 7 is a sectional diagram taken along line 7-7 of FIG. 3.

FIG. 8 is a diagram showing an implant holder and the surgical device shown in FIG. 1.

FIG. 9 is an isometric diagram of the surgical device shown in FIG. 1 installed between the end plates of two adjacent vertebrae.

FIG. 10 is a side view of the surgical device and adjacent vertebrae shown in FIG. 9.

### DETAILED DESCRIPTION

This disclosure relates to surgical implants having one or more strands extending from the implant to assist with the installation of the implants into patients, especially in minimally invasive surgical procedures. For example, the strand may be used during installation to provide a constant reference to the trailing edge of the implant in situ. In another example, the strand may be used during installation to provide guidance to the implant for instrumentation devices such as pusher instruments, attachment or removal instruments, etc. In another example, the strand may be used during installation to provide the ability to pull back on the implant. In another example, the strand may be grasped by an instrumentation device to help hold the implant to the instrumentation device. In another example, the strand allows for novel designs of installation and removal instruments. The strand may also be used in any other desired manner.

While the invention may be applied to any desired type of surgical implant, the invention will be described in the exemplary context of a transforaminal lumbar inter-body fusion (TLIF) device, which can be used in a spinal fusion procedure. The spine can be considered to be a series of movable segments made up of vertebrae and discs. Due to trauma, disease, and/or aging, the spine may be subject to degeneration. This degeneration may destabilize the spine and cause pain and/or nerve damage. Medical procedures are often required to either ease back pain, repair damage, or to prevent future damage. One procedure that is often used to treat back pain or spinal damage is spinal fusion. Spinal fusion is a surgical technique used to combine two or more adjacent vertebrae. Supplemental bone tissue is used in conjunction with the patient's natural osteoblastic processes in a spinal fusion procedure. Spinal fusion is used primarily to eliminate back pain caused by the motion of the damaged vertebrae by immobilizing adjacent vertebrae. Conditions for which spinal fusion might be done include degenerative disc disease, treatment of a spinal tumor, a vertebral fracture, scoliosis, degeneration of the disc, spondylolisthesis, or any other condition that causes instability of the spine.

One type of spinal fusion is interbody fusion. Typically, an interbody fusion procedure places a fusion cage and/or bone graft between the vertebra in the area normally occupied by an intervertebral disc. In preparation for a spinal fusion procedure, the intervertebral disc is removed. The end plates are then scraped to prepare the end plates for fusion. Scraping the end plates will disrupt the boney tissue, causing the tissue to bleed, heal, and fuse through the interbody fusion implant. An interbody device may be placed between the vertebra to maintain spine alignment and disc height. Fusion then occurs between the endplates of the vertebrae. In some examples, fusion is augmented by a process called fixation, meaning the placement of screws, rods or plates to stabilize the vertebra to facilitate bone fusion.

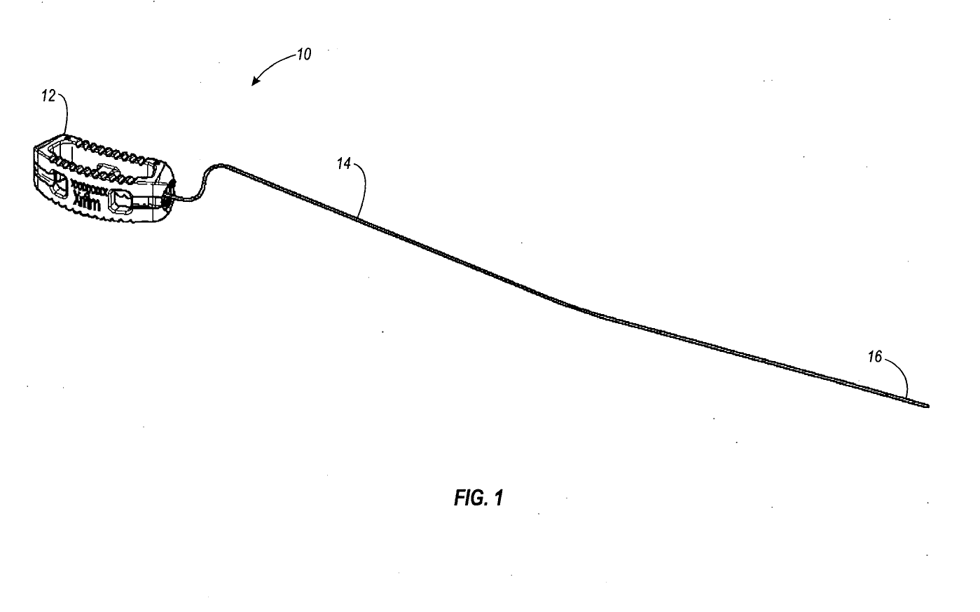

FIG. 1 is an isometric view of a surgical device 10, including an implant 12 and a strand 14 extending from the implant. In this example, the implant is a spacer configured to fit between adjacent vertebrae in a TLIF procedure. The strand 14 may be comprised of any desired material. Examples may include sutures, threads, wires, fibers, filaments, etc., or any other pliable and flexible material. In one example, the strand is made from a non-toxic hypoallergenic material. The strand can by absorbable, or nonabsorbable. One specific example of a strand is a #5 ethibond excel green braided polyester suture. In the example shown in FIG. 1, an optional leader 16 is formed at the end of the strand 14. The leader 16 can be stiffer than the rest of the strand to aid in insertion of the strand. The leader 16 may also keep the strand from fraying. The leader 14 may be any desired length.

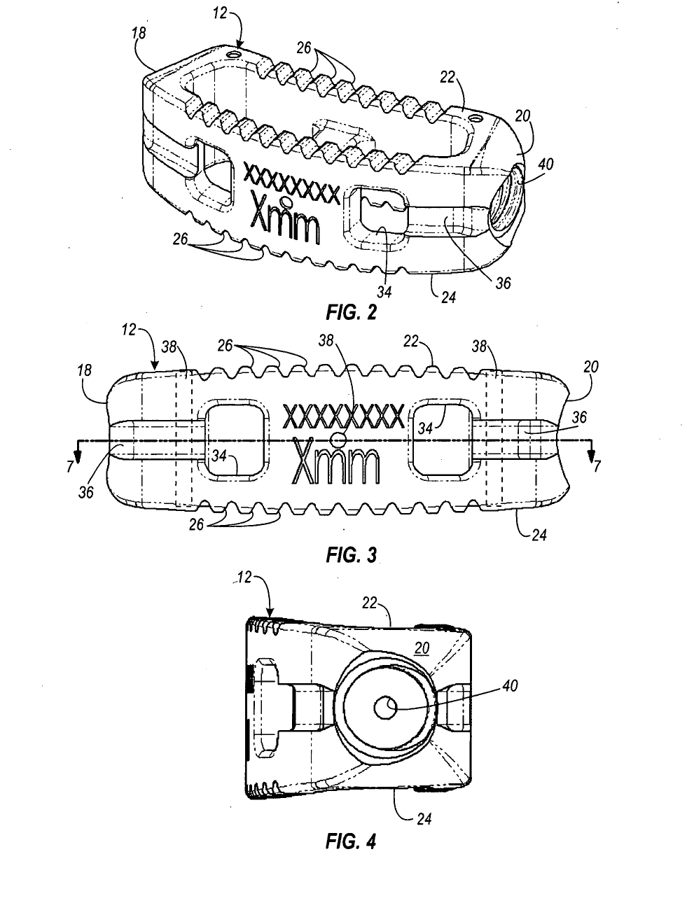

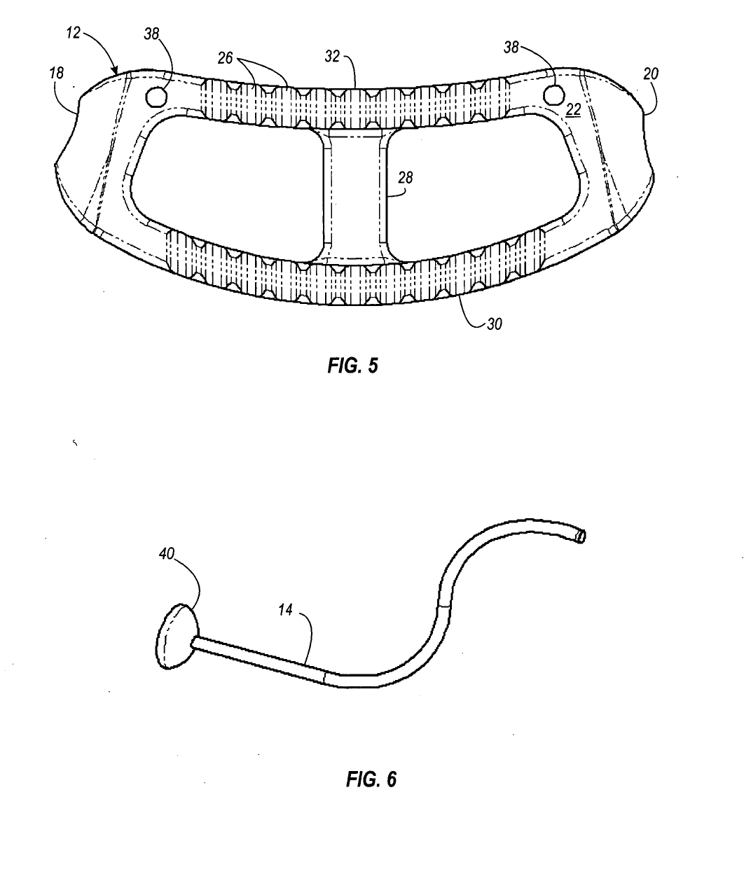

FIG. 2 is an enlarged isometric view of the implant 12 shown in FIG. 1. FIG. 3 is an enlarged front view of the implant 12 shown in FIG. 1. FIG. 4 is an enlarged side view of the implant 12 shown in FIG. 1. FIG. 5 is an enlarged top view of the implant 12 shown in FIG. 1. The implant 12 has opposing edges 18 and 20. In this example, either edge 18 or 20 may be coupled to the strand (discussed below). Also, either edge 18 or 20 may be considered to be the leading or trailing edge, depending on how the implant 12 is installed. The implant 12 also has top and bottom surfaces 22 and 24. The terms “top” and “bottom” are used for convenience only, and do not restrict the orientation of the implant 12. In the example shown, the implant 12 can be inserted with either surface 22 or 24 facing upward or downward relative to the patent. In the example shown in FIG. 3, the top and bottom surfaces 22 and 24 are curved, forming a dome-shape. The dome-shape assists in insertion of the implant, aids in mid-line location of the implant, and prevents post-op migration of the implant.

A plurality of ridges 26 are formed on the top and bottom surfaces 22 and 24 of the implant 12. The ridges 26 are configured to help hold the implant 12 to the end plates of the vertebrae to reduce the chance of medial/lateral migration of the implant 12. The implant 12 is generally hollow, as shown. A structural cross-bar 28 is formed between the front and back edges 30 and 32 of the implant 12 to strengthen the implant and minimize buckling upon insertion. A plurality of openings 34 are formed in the front edge 30 of the implant 12. The openings 34 allow bone fusion horizontally through the implant 12. The openings 24 may also function as implant holder features. If desired, similar openings could be formed in the back edge 32. The front and back edges 30 and 32 include horizontal grooves 36 formed to help an implant holder locate the implant superior/inferior and rotationally.

The implant 12 can be made from any desired materials. In one example, the implant is made from Polyetheretherketones (PEEK®) (or a similar material), bone, metal, or any other structural substitute. If the implant material is radio-lucent (such as with PEEK®), then doctors will be able monitor the fusion process better with X-rays. If desired, one or more radio opaque markers can be embedded into the implant, which will show up in an X-ray. The figures show three radio opaque markers 38 embedded into the implant at known locations. The markers 38 may be embedded in the implant at any desired locations. Since the positions of the markers are known relative to the fusion device, a doctor can determine the position of the fusion device in an X-ray by viewing the positions of the markers. In the example provided in the drawings (e.g., FIG. 3), two bar-shaped markers 38 extend between the top and bottom surfaces 22 and 24 near the posterior margin, and one bar-shaped marker 38 extends between the front and back surfaces 30 and 32 through the cross-bar 28.

An implant may be configured to any desired size or shape. In one example (in the example of a TLIF implant), the implant can be provided in multiple thicknesses, allowing a surgeon to select a desired size (e.g., 8 mm, 9 mm, 10 mm, 11 mm, 13 mm, 15 mm, etc.). In the example shown in the figures, the implant has about 5° of lordosis (e.g., see FIG. 4). The top and bottom surfaces 22 and 24 may also be parallel, or be formed at any other desired angle or shape. In the example shown in the figures, the implant has a lead-in width (e.g., see the edges 18 and 20 in FIG. 5) that is less than the width in the middle portion of the implant. In one example, the lead-in width is 9.5 mm, and the width in the middle portion of the implant is 11.0 mm.

FIG. 6 is a enlarged partial view of the strand 14 shown in FIG. 1. A retainer 40 is formed at the end of the suture, which inhibits the strand from sliding through the implant 12 (discussed below). Alternatively, a knot can be formed at the end of the strand.

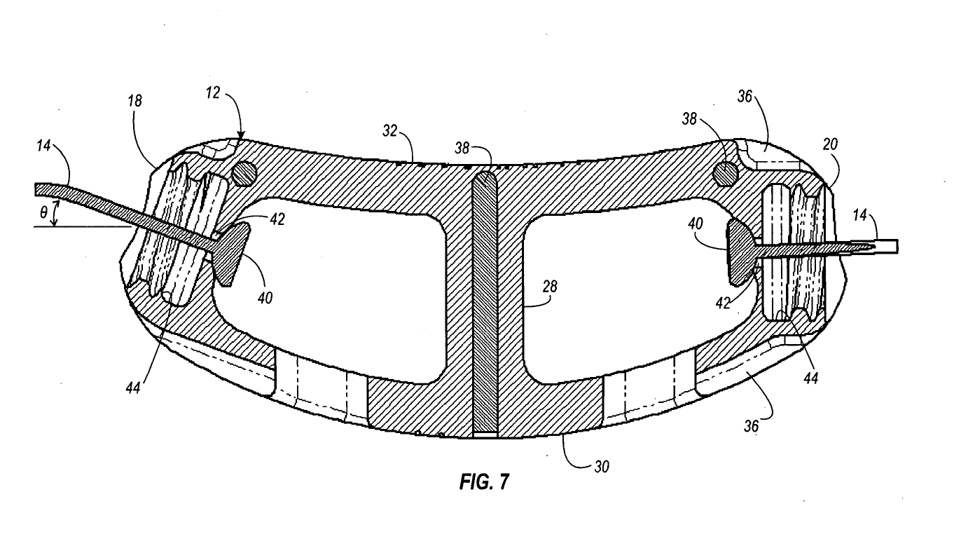

FIG. 7 is a sectional diagram taken along line 7-7 of FIG. 3. FIG. 7 also shows strands 14 inserted in alternate locations on the implant 12. At each edge 18 and 20 of the implant 12, a strand 14 is inserted through a hole 42 formed in the implant 12. The retainer 40 is sized to be larger than the holes 42 to hold the strand 14 in place. In the examples shown, the retainer 40 has a curved or spherical surface that generally matches a similar surface on the implant. Prior to installation of the implant 12, a strand 14 is inserted through one of the holes 42 and pulled through until the retainer 40 engages the implant.

FIG. 7 also illustrates how the strand 14 (and instrumentation devices) can be coupled to the implant 12 at different angles. The holes 42 are oriented at different angles relative to each other. In this example, the hole 42 at edge 20 is oriented generally parallel to the length of the implant 12. The hole 42 at edge 18 is oriented at an angle θ, relative to the length of the implant 12. In one example, θ is 20°, although any desired angle can be used. A surgeon can choose which hole 42 to use, as desired. Since, in this example, either surface 22 or 24 can be oriented up or down, a surgeon can choose either angle, regardless of which side of the patent the implant is being inserted.

FIG. 7 also illustrates examples of implant holder features. In this example, a threaded implant holder recess 44 is formed at each edge 18 and 20. As described above, the recesses 44 are oriented at different angles, allowing a surgeon to select between multiple attachment angles. In the example shown in FIG. 7, a surgeon can select from a neutral angle (by using the recess 44 at edge 20) or angle θ (by using the recess 44 at edge 18). The recesses 44 can be configured, as desired, with implant holders, or other instrumentation devices. In the example shown in FIG. 7, the recesses 44 are threaded to match corresponding instrumentation devices. Instrumentation devices may also be configured to engage the implant using the openings 34 and/or grooves 36.

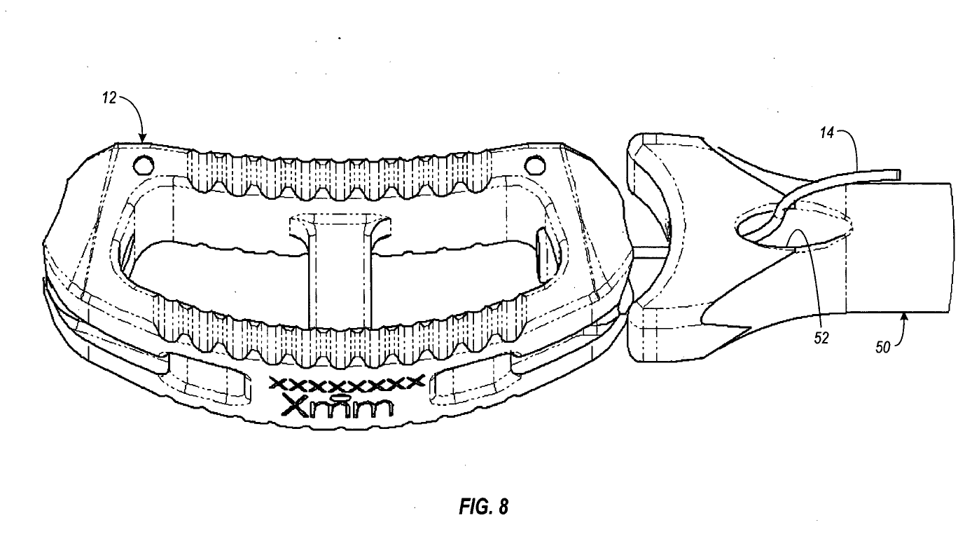

As discussed above, the strand 14 has several potential purposes, including providing guidance to instrumentation devices. FIG. 8 is a diagram showing an implant 12, strand 14, and a partial view of an implant pusher 50. The implant pusher 50 has an opening 52 adapted to receive the strand 14. After the implant 12 is inside a patient's body, the strand 14 can be used to guide the implant pusher 50 to the implant 12.

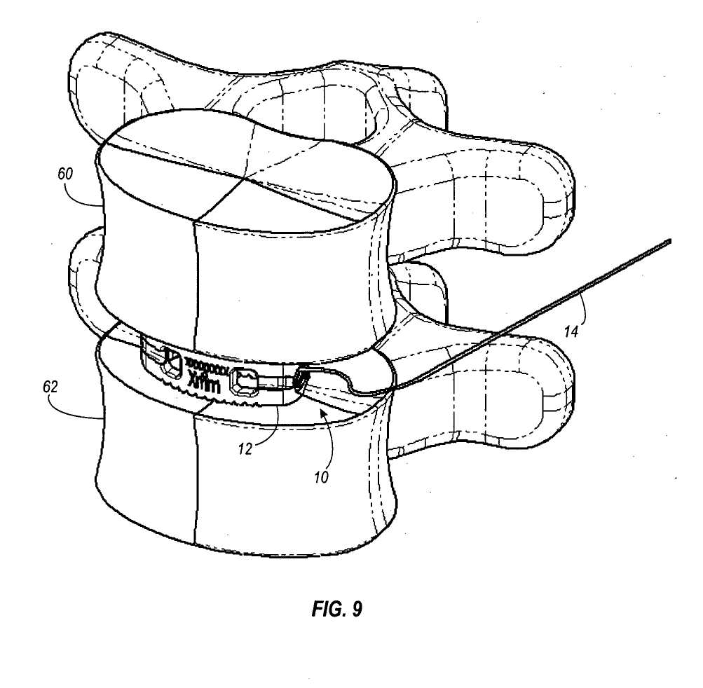

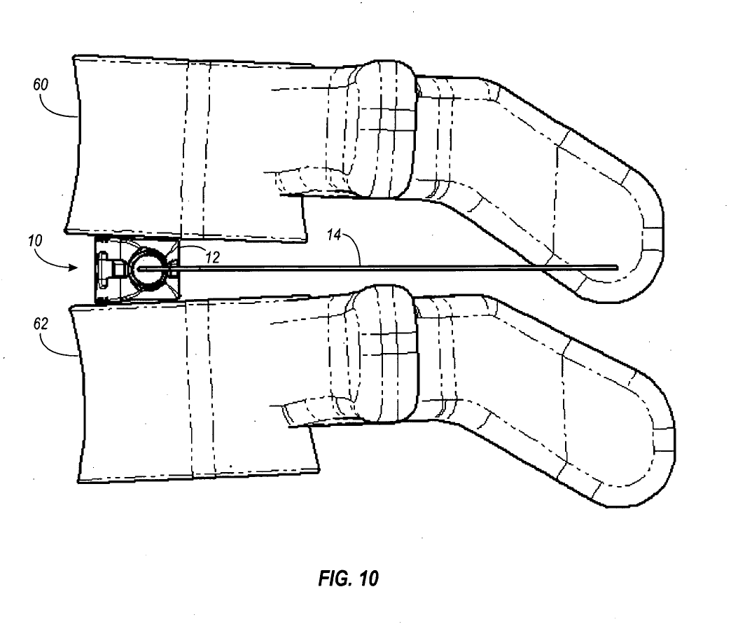

FIG. 9 is an isometric diagram of the surgical device 10 shown in FIG. 1 installed between the end plates of two adjacent vertebrae 60 and 62 to facilitate the fusion of the vertebrae 60 and 62. FIG. 10 is a side view of the surgical device 10 shown in FIG. 9. For clarity, FIGS. 9 and 10 do not show other components that might be used in a TLIF procedure, such as fusion material, pedicle screws and rods, etc. The implant 12 provides load bearing support as well as the proper spacing between the vertebrae 60 and 62 while fusion of the vertebrae takes place. The implant 12 is positioned between the end plates of the vertebrae 50 and 52 within the vertebral body in the area usually occupied by the intervertebral disc. For clarity, the disc annulus is not shown, so the position of the implant 12 can be seen. FIGS. 9 and 10 also shows the strand 14 extending from the implant 12.

Following is an exemplary description outlining the use of a suture guided implant, in the context of a TLIF procedure. In one example, a window is cut in the side of the disc annulus to allow a fusion cage (e.g., implant 12) to be inserted. The nucleus pulposus can also be cleaned out to provide room for the implant. Prior to installation of the implant, the vertebral bodies are prepared (e.g., by scraping, etc.) for the implant. Next, a desired implant is selected. The selection of an implant can be based on factors such as the desired height between the adjacent vertebrae, the desired lordosis, etc. Next, the implant is placed in the desired position between the vertebral bodies. Any desired fusion material (i.e., a material that will promote fusion, etc.) may be packed between the vertebral bodies and inside the implant. Any other desired components are also installed (e.g., pedicle screws and rods, bone plates, etc.). During the installation process, the strand may be used in several ways. For example, the surgeon can guide instrumentation devices to the implant, using the strand as a guide. The implant can be pulled back, if needed, by pulling on the strand. The strand can also be used as a reference to the trailing edge of the implant, insitu. The strand may also be used in any other desired manner. Once the implant is installed, the strand may be cut off, if desired.

In the preceding detailed description, the embodiments of the invention are described with reference to specific exemplary embodiments thereof. Various modifications and changes may be made thereto without departing from the broader spirit and scope of the invention as set forth in the claims. The specification and drawings are, accordingly, to be regarded in an illustrative rather than a restrictive sense.

What is claimed is:

1. A surgical implant comprising:
an implant; and
a strand coupled to and extending from the implant, wherein the strand is configured to provide guidance for surgical instruments that are used during the installation of the impant.

2. The surgical implant of claim 1, wherein an opening is formed in the implant for receiving the strand.

3. The surgical implant of claim 2, wherein one end of the strand is enlarged to retain the strand to the implant.

4. The surgical implant of claim 2, wherein a second opening is formed in the implant for receiving the strand, allowing a user to select where the strand is coupled to the implant.

5. The surgical implant of claim 4, wherein the implant is configured to be coupled to one or more instrumentation devices proximate the first and second openings.

6. The surgical implant of claim 4, wherein the angle of an instrumentation device, relative to the implant, is different, depending on whether the instrumentation device is coupled to the implant proximate the first or second openings.

7. A spinal fusion device comprising:
a spacer configured to be placed between adjacent vertebrae; and
a strand coupled to the implant, wherein the strand is configured to provide guidance for surgical instruments that are used during the installation of the implant.

8. The spinal fusion device of claim 7, wherein an opening is formed in the spacer for receiving the strand.

9. The spinal fusion device of claim 8, wherein one end of the strand is enlarged to retain the strand to the spacer.

10. The spinal fusion device of claim 8, wherein a second opening is formed in the spacer for receiving the strand, allowing a user to select where the strand is coupled to the spacer.

11. The spinal fusion device of claim 10, wherein the spacer is configured to be coupled to one or more instrumentation devices proximate the first and second openings.

12. The spinal fusion device of claim 10, wherein the angle of an instrumentation device, relative to the spacer, is different, depending on whether the instrumentation device is coupled to the spacer proximate the first or second openings.

13. The spinal fusion device of claim 7, further comprising a plurality of ridges formed in upper and lower opposing surfaces of the spacer to prevent migration of the spacer.

14. The spinal fusion device of claim 7, wherein the spacer is comprised of a thermoplastic material.

15. The spinal fusion device of claim 7, wherein the spacer is comprised of Polyetheretherketones (PEEK).

16. The spinal fusion device of claim 7, further comprising one or more radio opaque markers formed in the spacer to allow a user to determine the position of the spinal fusion device relative to a spine using x-rays.

17. The spinal fusion device of claim 7, wherein the spinal fusion device is a transforaminal lumbar inter-body fusion (TLIF) device.

18. A method of installing a surgical implant comprising:
providing an implant coupled to a strand that extends from the implant;
using the strand to guide one or more instrumentation devices to the implant; and
using the one or more instrumentation devices to position the implant where desired.

19. The method of claim 18, wherein the implant is a spinal fusion device, the method further comprising inserting the implant between two adjacent vertebrae.

20. The method of claim 19, wherein the strand is used to guide the one or more instrumentation devices to the implant after the implant has been inserted between the two adjacent vertebrae.
